(19) United States
(12) Patent Application Publication
(10) Pub. No.: US 2006/0216255 A1
(43) Pub. Date: Sep. 28, 2006

(54) Medicinal cosmetical composition with areca catechu seed extract

(76) Inventors: Kun-Kook Lee, Cheongju (KR); Kwang-Sik Lee, Cheonan (KR)

Correspondence Address: NIXON & VANDERHYE, PC, 901 NORTH GLEBE ROAD, 11TH FLOOR, ARLINGTON, VA, 22203, US

(21) Appl. No.: 10/553,261

(22) PCT Filed: Apr. 11, 2003
(86) PCT No.: PCT/KR03/00733

§ 371 Date: Dec. 2, 2005

(51) Int. Cl. A61K 8/00

(52) U.S. Cl. 424/70.1

(57) ABSTRACT

The present invention relates to a composition for promoting the proliferation of fibroblasts and kerathincytes comprising a mixed extract from Areca catechu seed and Glycyrrhiza glabra and a cosmetic composition for skin-whitening and a remedy of skin wrinkles comprising the same.

### BACKGROUND OF THE INVENTION

1. Field of the Invention

The present invention relates to a composition for promoting fibroblasts and karatinocytes proliferation and a cosmetic composition for skin whitening and remedy of skin wrinkles, more particularly, to a composition for promoting the proliferation of fibroblasts and karatinocytes comprising a mixed extract from Areca catechu seed and Glycyrrhiza glabra and a cosmetic composition comprising the same.

2. Description of the Related Art

A skin, the largest organ in human body, is considered to be pivotal because it is involved in a variety of physiological functions such as protection of several organs from environmental stimulations, offering a barrier for prevention water and useful constituents in body from release, regulation of body temperature, respiration and excretion.

However, with aging, the thickness of epidermis, dermis and subdermal tissues is decreased to result in the declination of barrier function of skin, so that the physiological functions of skin are lowered. As a result, skin aging comes to appear.

The physiological alterations in skin, with aging, include: (a) decrease of the thickness of the epidermis, dermis and subdermal tissues; (b) decrease of barrier function due to alteration of lipid composition and content in lipid barrier resulting in skin drying; and (c) elicitation of freckles, pigmentation and various skin lesions.

Furthermore, the active oxygen and free radicals elicited by ultraviolet ray, air pollution or severe stress may oxidize or denature the constituents (e.g. protein, nucleic acid and membrane lipid) of human body, serving as main factor of skin aging. Therefore, the researches in the cosmetic art have been made to prevent and treat the skin aging-associated phenomena such as wrinkles, decreased elasticity, pigmentation, freckles and drying. Among such researches, the remedy of skin wrinkles is partially successful in a cosmetic composition.

Japanese Unexamined Publication Pyeong 5-246838 discloses the method for remedying skin wrinkles through promotion of collagen production, suggesting that skin wrinkles are ascribed predominantly to decomposition of collagen and elastin with aging.

Elastin fiber in skin forms cross-linkage together with collagen fiber in epidermis. With the progress of aging, the action of elastin-degradable enzyme, elastase, is responsible for the sharp declination of skin elasticity, causing sagging. In addition, with aging, in view of histology, the infiltration of phlogocyte occurs frequently, the lack and aggregation of elastin fiber is induced and the amount of collagen fiber is reduced and in view of biochemistry, the activity of elastase is remarkably decreased. Elastase has been reported to be a sole enzyme catalyzing degradation of elastin and thus the inhibition of its activity or generation could be considered to be a fundamental approach for lessening skin aging.

In order to retard skin aging, the cosmetic compositions conventionally contain moisturing agent, anti-inflammatory agent or nutritive additives for tissues with degraded cross-linkages of elastin and collagen. However, such compositions generally exhibit a limitation in the fundamental retardation of skin aging. Therefore, there remains a need in the art for an inhibitor capable of fundamentally preventing the degradation of elastin and collagen.

Endeavoring to resolve the limitation of the conventional agents for remedying skin aging, the present applicant has found and reported that the extract obtained from seeds of Areca catechu, having been served as oriental medicine in Korea, exhibited a remarkable inhibition effect to elastase and could function as a free-radical scavenger, thereby being able to prevent skin aging (see, Korean Pat. Appln. Nos. 1997-78817 and 1999-56924; and Int. J. Cosmet. Sci., 21:275-294(1999)).

The color of human skin is ascribed mainly to the amounts of melanin, karatin and hemoglobin. The melanin is considered as a pivotal factor for skin color. Although the melanin functions in determination of skin color and protection of skin by serving as an absorbent of ultraviolet ray and a free radical scavenger, it causes pigmentation in skin leading to skin-darkening and generation of freckles when over-expressed in skin due to environmental changes (e.g. over-exposure to ultraviolet ray, air pollution and mental stress).

The detail mechanism of melanin synthesis is as follows: In a melanocyte, a tyrosinase converts tyrosine into dopaquinone and it undergoes autoxidation and enzymatic reactions, finally producing a copolymer, melanin. The melanin thus generated is transferred to keratinocyte through melanosome and then undergoes keratinization for about 28 days, finally secreted to skin surface. Where the melanin is over-produced due to a factor for promoting melanin generation and is completely removed by virtue of keratinization, the pigmentation appears. Accordingly, the adjustment of a process involved in melanin generation makes it possible to prevent pigmentation.

Several approaches have been suggested to inhibit melanin generation. Their representative is to inhibit tyrosinase, an essential enzyme for melanin synthesis. A chelator, kojic acid and a substrate-like substance, arbutin has been revealed to show an inhibitory activity to tyrosinase. In addition, plant extracts from Morus alba and Glycyrrhiza glabra has been also reported such inhibitory activity (Jpn J. Dermatol. 102:679-689(1992)). However, the skin-whitening agents described above exhibit poor stability, so that its long action cannot be expected and thus its application range to cosmetics is very limited.

Throughout this application, various patents and publications are referenced and citations are provided in parentheses. The disclosure of these patents and publications in their entities are hereby incorporated by references into this application in order to more fully describe this invention and the state of the art to which this invention pertains.

### SUMMARY OF THE INVENTION

The present inventors have made intensive research to develop a novel cosmetic composition with dual function for skin-whitening and remedy of skin wrinkles, it has been found that a mixed extract from Areca catechu seed and Glycyrrhiza glabra exhibited the dual function in a synergic manner. In addition, it has been discovered that where the mixed extract was entrapped into a suitable vesicular structure, the effects and stability of the two active ingredients were dramatically enhanced.

Accordingly, it is an object of this invention to provide a composition for promoting fibroblasts and karatinocytes proliferation.

It is another object of this invention to provide a composition for promoting the production of integrin in fibroblasts.

It is still another object of this invention to provide a cosmetic composition with dual function for skin whitening and remedy of skin wrinkles.

Other objects and advantages of the present invention will become apparent from the detailed description to follow and together with the appended claims and drawings.

### DETAILED DESCRIPTION OF THIS INVETNION

In one aspect of this invention, there is provided a composition for promoting fibroblasts and karatinocytes proliferation, comprising an extract from a seed of Areca catechu and an extract from Glycyrrhiza glabra.

The present inventors have found that the mixed extract from Areca catechu seed and Glycyrrhiza glabra used as an oriental medicine in Korea was very effective in proliferation of fibroblasts and karatinocytes that are very closely related to the remedy of skin wrinkles.

Areca catechu is widely distributed and cultivated in several regions such as South China, Taiwan and Malaysia. The seed of Areca catechu has been medically used in the Orient for treating dyspepsia, constipation and stomachache (see Japanese Unexamined Publication Pyeong 5-320037). In addition, the present applicant has reported that Areca catechu seed exhibited a remarkable inhibition effect to elastase and could function as a free-radical scavenger, thereby being able to prevent skin aging (see, Korean Pat. Appln. Nos. 1997-78817 and 1999-56924; and Int. J. Cosmet. Sci., 21:275-294(1999)).

Glycyrrhiza glabra is a perennial herb belonging to Leguminosae, and has been suggested to show anti-inflammatory activity, anti-allergic activity, antibiotic activity and skin-whitening effect.

In a preferred embodiment, the extracts from Areca catechu seed and Glycyrrhiza glabra are obtained using various extraction solvents: (a) water, (b) absolute or water-bearing lower alcohol containing 1-4 carbon atoms (methanol, ethanol, propanol, butanol, etc.), (c) mixture of lower alcohol and water, (d) acetone, (e) ethyl acetate, (f) chloroform, (g) 1,3-butylene glycol and (h) butyl acetate. Furthermore, it is apparent to one skilled in the art that other conventional solvents may be employed for substantially similar extraction efficiency.

The extracts from Areca catechu seed and Glycyrrhiza glabra can be purified using the well-known methods in the art. For instance, an ultrafiltration with defined molecular weight cut-off value and various chromatography (for purification dependent upon size, charge, hydrophobicity and affinity) may be used for obtaining the extracts from Areca catechu seed and Glycyrrhiza glabra. In addition, the gas chromatography, head space gas chromatography, liquid chromatography, high performance liquid chromatography and thin layer chromatography may be used for this invention.

The extracts from Areca catechu seed and Glycyrrhiza glabra can be obtained in a form of powder by use of lyophilization and spray drying.

Each of Glycyrrhiza glabra extract and Areca catechu seed extract can enhance the proliferation of fibroblasts and kerationcytes that has been known to play an important role in remedy of skin wrinkles and improvement of skin elasticity. It is notable that the mixed extract containing Glycyrrhiza glabra extract and Areca catechu seed extract exhibits a synergic effect on the proliferation of fibroblasts and kerationcytes. The proliferation of fibroblasts and kerationcytes is closely related to biosynthesis of collagen, elastin, integrin and laminin that are pivotal proteins for remedy of skin wrinkles and improvement of skin elasticity.

In another aspect of this invention, there is provided a composition for enhancing the integrin production in fibroblasts, which comprises an Areca catechu seed extract as active ingredient.

The integrin is a connective protein for promoting a signal transmission between cells and cell activity by enhancing the connection between cells. The Areca catechu seed extract is very successful in promoting the integrin production in fibroblasts. It will be appreciated that where the Areca catechu seed extract acts on fibroblasts playing a pivotal role in determination of skin condition, the increased integrin allows to increase the activity of fibroblasts, thereby making it possible to remedying skin wrinkles.

In still another aspect of this invention, there is provided a cosmetic composition with dual function for skin whitening and remedy of skin wrinkles comprising: (a) an extract from a seed of Areca catechu and an extract from Glycyrrhiza glabra as an active ingredient; and (b) a cosmetically acceptable carrier.

According to the findings of the present inventors, the Areca catechu seed extract can synergistically improve the effects of Glycyrrhiza glabra extract: the inhibition on tyrosinase activity and inhibition on melanin synthesis in melanocyte. The skin-whitening effect of the present composition could be synergistically increased in comparison to that of Glycyrrhiza glabra extract alone, which is considered one of features of this invention.

As described previously, the enhancement effect of the Areca catechu seed extract on the production of integrin is partially responsible for treating skin wrinkles of the present composition.

In addition, the mixed extract from Glycyrrhiza glabra and Areca catechu seed contained in the present cosmetic composition can improve the proliferation of fibroblasts and kerationcytes, which is also partially responsible for treating skin wrinkles of the present composition.

Therefore, the present cosmetic composition is accomplished based on the novel and unobvious findings: (a) Compared to single use of Glycyrrhiza glabra extract and Areca catechu seed extract, their mixture exhibits enhanced own effect (skin-whitening and skin wrinkle treatment) in synergistic manner; (b) The Areca catechu seed extract is capable of enhancing integrin production in fibroblasts; (c) the mixed extract from Glycyrrhiza glabra and Areca catechu seed is very successful in promoting the proliferation of fibroblasts and kerationcytes; and (d) the instability of Glycyrrhiza glabra extract in a cosmetic composition may be solved by the aid of Areca catechu seed extract.

According to a preferred embodiment, each of the extract from the seed of Areca catechu and the extract from Glycyrrhiza glabra is present in an amount of 0.001-1.0 wt % based on the total weight of said composition, more preferably, 0.002-0.5 wt %.

In a preferred embodiment, the mixed extract from Glycyrrhiza glabra and Areca catechu seed is entrapped into a vesicular structure. The vesicular structure described herein includes liposome, noisome, biosome and pharmacosome. Most preferably, the carrier for application to skin is a biosome.

The most suitable carrier, biosome, may improve stability and skin penetration of two active ingredients (Glycyrrhiza glabra extract and Areca catechu seed extract) and thus highly increase the skin-whitening effect and remedy effect of skin wrinkles, thereby highly shortening the time period for exhibiting the effects in a practical use.

According to a preferred embodiment, the biosome is present in an amount of 0.05-20 wt % based on the total weight of the composition, more preferably, 0.1-10.0 wt %.

The biosome carrier used in this invention may be prepared according to the conventional methods known to one skilled in the art. Preferably, the non-ionic surfactant is used, including polyoxyethylene alkylether, polyoxyethylene cholesterylether, polyoxyethylene sorbitanester, polyglyceryl alkylester, polyoxyethylene alkylester and sugar diester. The suitable amount of non-ionic surfactant ranges from 10-50 wt % based on the total weight of the biosome, more advantageously, 30-40 wt %.

According to a preferred embodiment, the non-ionic surfactant is used together with its base, e.g. PEG-5-soy sterol and cholesteryl oleate.

Furthermore, since the non-ionic surfactant may not exhibit similar function to that of lipid derived from skin, it is preferred that the composition for preparing biosome further contains a ceramide bound to a glycerin skeleton. The ceramide bound to a glycerin skeleton has an amphiphilicity, so that it exhibits a similar property to that of natural-occurring lipid of cell membrane. It is preferred that the amount of ceramide ranges from 0.1-10 wt % based on the total weight of biosome, more preferably, 2.0-6.0 wt %.

Preferably, the type of the ceramide is ceramide 3 and pseudoceramide represented by the following formula I:

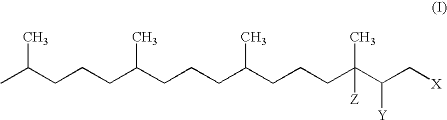

wherein Z represents —OH and Y represents —OH, 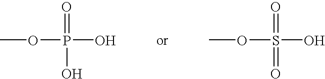
with the proviso that X is 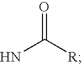
Z represents —OH and X represents —OH, 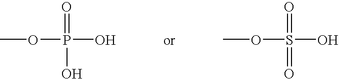
with the proviso that Y is 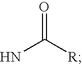
Y represents —OH and X represents —OH, 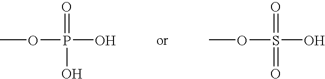
with the proviso that Z is 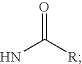
R represents a linear or branched, saturated or unsaturated aliphatic hydrocarbon group; and when substituted, R has one or more —OH groups.

More preferably, the pseudoceramide represented by the formula I is one represented by the following formula II, III and IV:

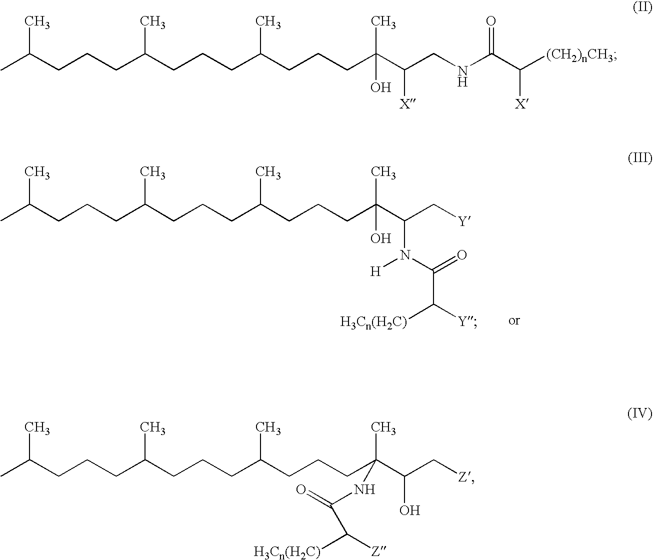

wherein X′ represents H or —OH; X″ represents —OH, 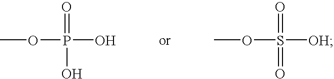
Y′ represents —OH, 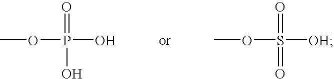
Y″ represents H or —OH; Z′ represents —OH, 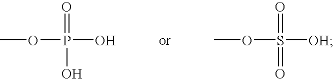
Z″ represents H or —OH; and n is 0 or an integer of from 1 to 47.

The specific examples and methods for preparing pseudoceramides described above are disclosed in PCT/KR02/00314, which is incorporated herein by reference. The pseudoceramides show the identical functions to those of natural-occurring ceramide and higher solubility, so that their applicability to cosmetic composition is excellent. In addition, the pseudoceramides may be prepared in cost-effective manner.

According to a preferred embodiment, the composition for preparing biosome further contains co-emulsifier such as monoglycerol, diglycerol and triglycerol. Diglycerol is the most preferred. It is preferred that the amount of co-emulsifier is from 1 to 15 wt % based on the total weight of biosome, more preferably, 2-10 wt %.

The composition for preparing biosome, preferably, contains cholesterol alkylester such as cholesteryl nonanoate, cholesteryl stearate, cholesteryl isostearate and cholesteryl isostearylcarbonate.

It is preferred that the extract from the seed of Areca catechu and the extract from Glycyrrhiza glabra are entrapped into the biosome with an amount of 0.01-5.0 wt % based on the total weight of the biosome.

Furthermore, the cosmetic compositions of the present invention may contain auxiliaries as well as carrier in addition to Areca catechu seed extract and Glycyrrhiza glabra extract (or biosome carrying Areca catechu seed extract, Glycyrrhiza glabra extract).

The non-limiting examples of auxiliaries include preservatives, antioxidants, stabilizers, solubilizers, vitamins, colorants, odor improvers or mixtures of these ingredients.

The cosmetic compositions of this invention may be formulated in a wide variety of form, for non-limited example, including a solution, a suspension, an emulsion, a paste, an ointment, a gel, a cream, a lotion, a powder, a soap, a surfactant-containing cleanser, an oil, a powder foundation, an emulsion foundation, a wax foundation and a spray. In detail, the cosmetic composition of the present invention can be provided in a form of skin softener (skin lotion), astringent lotion, nutrient emulsion (milk lotion), nutrient cream, message cream, essence, eye cream, cleansing cream, cleansing foam, cleansing water, facial pack, spray or powder.

The cosmetically acceptable carrier contained in the present cosmetic composition, may be varied depending on the type of the formulation. For example, the formulation of ointment, pastes, creams or gels may comprise animal and vegetable fats, waxes, paraffins, starch, tragacanth, cellulose derivatives, polyethylene glycols, silicones, bentonites, silica, talc, zinc oxide or mixtures of these ingredients.

In the formulation of powder or spray, it may comprise lactose, talc, silica, aluminum hydroxide, calcium silicate, polyamide powder and mixtures of these ingredients. Spray may additionally comprise the customary propellants, for example, chlorofluorohydrocarbons, propane/butane or dimethyl ether.

The formulation of solution and emulsion may comprise solvent, solubilizer and emulsifier, for example water, ethanol, isopropanol, ethyl carbonate, ethyl acetate, benzyl alcohol, benzyl benzoate, propylene glycol, 1,3-butyleneglycol, oils, in particular cottonseed oil, groundnut oil, maize germ oil, olive oil, castor oil and sesame seed oil, glycerol fatty esters, polyethylene glycol and fatty acid esters of sorbitan or mixtures of these ingredients.

The formulation of suspension may comprise liquid diluents, for example water, ethanol or propylene glycol, suspending agents, for example ethoxylated isosteary alcohols, polyoxyethylene sorbitol esters and poly oxyethylene sorbitan esters, micocrystalline cellulose, aluminum metahydroxide, bentonite, agar and tragacanth or mixtures of these ingredients.

The formulation of cleansing compositions with surfactant may comprise aliphatic alcohol sulfate, aliphatic alcohol ether sulfate, sulfosucinnate monoester, isothinate, imidazolium derivatives, methyltaurate, sarcocinate, fatty acid amide ether sulfate, alkyl amido betain, aliphatic alcohol, fatty acid glyceride, fatty acid diethanolamide, vegetable oil, lanoline derivatives, ethoxylated glycerol fatty acid ester or mixtures of these ingredients.

The following specific examples are intended to be illustrative of the invention and should not be construed as limiting the scope of the invention as defined by appended claims.

### EXAMPLES

### Preparatory Example I

### Preparation of Biosome

The biosomes used in the present Examples were prepared in such a manner that non-ionic surfactant, co-emulsifier, cholesterol, cholesterol ester and soy sterol were emulsified under high pressure to give a biosome with closed bilayer structure. Two active ingredients, Areca catechu seed extract, Glycyrrhiza glabra extract were entrapped into the prepared biosome, so that they are stabilized and much more effective.

40 wt % of polyoxyethylene alkylether, 25 wt % of PEG-5-soy sterol and cholesteryl oleate as base of non-ionic surfactant, 3.0 wt % of ceramide 3 bound to glycerin, 5.0 wt % of diglycerol as co-emulsifier, cholesteryl stearate and residual amount of distilled water were heated and mixed at 75° C. Then, each 3 wt % of Areca catechu seed extract and Glycyrrhiza glabra extract were added to the mixture and emulsified under a high pressure of 1,000 bar, thereby yielding biosome with a particle size of 100 nm.

### Preparatory Example II

### Preparation of Extract from Seeds of Areca catechu

Seeds of Areca catechu were washed with distilled water and dried. One kg of the dried seeds was added to 5 L of 70% ethanol and underwent extraction for 5 days at 4-40° C. The extract was filtered through 300 mesh filter cloth and stood for 7-10 days at 5-10° C., followed by filtering through Whattman No. 5 filter paper. The filtrate was dried in a rotary vacuum evaporator to yield a power of the extract from seed of Areca catechu. 100 g of a power of Areca catechu seed extract were dissolved into 1 L of a mixture of the same volume of water and 1,3-butylene glycol to obtain a solution of Areca catechu seed extract.

The amounts of the extract from seed of Areca catechu described herein are indicated as a concentration of the powder.

### Preparatory Example III

### Preparation of Extract from Glycyrrhiza glabra

Glycyrrhiza glabra were washed with distilled water and dried. One kg of the dried Glycyrrhiza glabra was added to 5 L of 70% ethanol and underwent extraction for 5 days at 4-40° C. The extract was filtered through 300 mesh filter cloth and stood for 7-10 days at 5-10° C., followed by filtering through Whattman No. 5 filter paper. The filtrate was dried in a rotary vacuum evaporator to obtain a power of Glycyrrhiza glabra extract.

### EXAMPLE AND COMPARATIVE EXAMPLE

TABLE I

| Ingredients | Example | Com. Exam 1 | Com. Exam 2 |
|---|---|---|---|
| Biosome* | 10.0 | — | — |
| Areca catechu seed extract | | 0.5 | — |
| Glycyrrhiza glabra extract | | 0.5 | — |
| Cetostearyl alcohol | 2.0 | 2.0 | 2.0 |
| Liquid paraffin | 3.0 | 3.0 | 3.0 |
| Delta-tocopherol | 0.2 | 0.2 | 0.2 |
| Glyceryl stearate | 1.5 | 1.5 | 1.5 |
| Polysorbate 60 | 1.2 | 1.2 | 1.2 |
| Sorbitan cesquinoleate | 0.5 | 0.5 | 0.5 |
| Squalane | 5.0 | 5.0 | 5.0 |
| Cyclomethicone | 3.0 | 3.0 | 3.0 |
| Microcrystalline | 0.7 | 0.7 | 0.7 |
| Trioctanoine | 5.0 | 5.0 | 5.0 |
| BHT | 0.05 | 0.05 | 0.05 |
| 1,3-butylene glycol | 2.0 | 2.0 | 2.0 |
| Conc. Glycerine | 4.0 | 4.0 | 4.0 |
| EDTA-2Na | 0.05 | 0.05 | 0.05 |
| Santan gum | 0.1 | 0.1 | 0.1 |
| Tocopheryl acetate | 0.2 | 0.2 | 0.2 |
| Perfume, preservative | 0.3 | 0.3 | 0.3 |
| Distille water | To 100 | To 100 | To 100 |

*containing --- wt % of Areca catechu seed extract and --- wt % of Glycyrrhiza glabra extract
The numerals in Table indicate the amounts of the ingredients by weight % based on the total weight of the composition.

### Experimental Example I

### Analysis of Inhibition Effect on Tyrosinase Activity

The tyrosinase inhibition effects of Areca catechu seed extract, Glycyrrhiza glabra extract and a mixture thereof were examined.

A tyrosinase commercially available from Sigma Co, which is separated and purified from mushroom, was employed. As substrate, a tyrosine was dissolved in 0.05 M sodium phosphate buffer (pH 6.8) at a concentration of 0.1 mg/ml.

Each extract (in the form of powder) was dissolved in 1,3-butylene glycol at a high concentration, and the solution was further diluted to an appropriate concentration with a buffer solution, followed by mixing its same volume to give the present mixed extract.

Tyrosine solution (0.5 ml) was placed in a test tube and the extract (0.5 ml) was added thereto. The test tube was stood in an incubator at 37° C. for 10 min, and then 200 U/ml tyrosinase (0.5 ml) was added thereto. The reaction was carried out at the same temperature for 10 min. As a control, either Areca catechu seed extract or Glycyrrhiza glabra extract (0.5 ml) was used instead of the mixed extract. The reaction was stopped by placing the test tube containing the reactant on ice. Absorbance was measured at a wavelength of 475 nm by use of spectrophotometer.

The inhibition effects on tyrosinase activity were determined by the equation below:
Inhibition ratio on tyrosinase activity (%)=100−[(absorbance of each extract/absorbance of control)×100]

The results are shown in Table II.

TABLE II

| | Inhibition ratio on tyrosinase activity (%) | | |
|---|---|---|---|
| | | | Glycyrrhiza glabra extract |
| Conc. | Glycyrrhiza | Areca catechu | (50%) + Areca catechu seed |
| (μg/ml) | glabra extract | seed extract | extract (50%) |
| 10 | 15.8 | 4.4 | 30.1 |
| 20 | 27.5 | 7.2 | 37.5 |
| 50 | 36.8 | 14.6 | 59.5 |
| 100 | 44.4 | 18.8 | 81.9 |
| 200 | 62.2 | 22.3 | 97.3 |

As shown in Table II, the inhibition effect of Glycyrrhiza glabra extract on tyrosinase activity was synergistically increased by virtue of Areca catechu seed extract. It is novel and surprising that Areca catechu seed extract can promote the inhibition effect of Glycyrrhiza glabra extract on tyrosinase activity. Therefore, it could be understood that the inhibition on tyrosinase activity, i.e., skin-whitening effect, is accomplished at a desired level even when the less amount of the mixed extract is incorporated into a cosmetic composition compared to the conventional ingredients for skin-whitening such as Glycyrrhiza glabra extract.

### Experimental Example II

### Analysis of Inhibition Effect on Melanin Synthesis in Melanocyte

As melanocyte, the commercially available B-16 melanoma cell line derived from mouse (ATCC CRL 6323) was employed. The melanoma cell line was inoculated in DMEM containing 4.5 g/L glucose, 10% fetal bovine serum and 1% penicillin-streptomycin, and cultivated in a 50 ml T-flask at 37° C. After cultivating under 5% CO₂ for 24 hr, the culture solution was treated with 0.05% trypsin containing 0.02% EDTA to detach cells and then cultivated for additional 48 hr. At this time, the number of cells was 5.76×10⁶ cells/flask. A diluted solution of the mixed extract in DMEM was added to the cultivated melanoma cells and then cultivated at 37° C. for 5 days.

After cultivation, the culture medium was discarded and the residual was treated with 1 ml of phosphate buffered saline (PBS) containing 0.02% EDTA and 0.05% trypsin to detach cells, followed by centrifugation for 5 min to collect cells. The collected cells were treated with trichloroacetic acid, agitated, and centrifuged. The melanin precipitated was washed with PBS and treated with 1 N NaOH to dissolve melanin. Absorbance at 475 nm was measured. Melanin concentration was determined from standard concentration curve of synthetic melanin (available from Sigma Co.). The results are shown in Table III.

TABLE III

Inhibition ratio on melanin synthesis (%)

| | | Mixed extract |
|---|---|---|
| Extract | | Glycyrrhiza glabra extract |
| concentration | Glycyrrhiza | (50%) + Areca catechu seed |
| (μg/ml) | glabra extract | extract (50%) |
| 10 | 16.8 | 27.4 |
| 20 | 21.2 | 31.4 |
| 50 | 29.8 | 43.7 |
| 100 | 32.1 | 54.2 |

As indicated Table III, the inhibition effect of Glycyrrhiza glabra extract on melanin synthesis was synergistically increased by virtue of Areca catechu seed extract. It is novel and surprising that Areca catechu seed extract can enhance the inhibition effect of Glycyrrhiza glabra extract on melanin synthesis. These results correspond to those in Experimental Example I.

### Experimental Example III

### Analysis of Inhibition Effect on Elastase Activity

For this analysis, a porcine pancreatic elastase was purchased from Sigma Co. 1 ml of a substrate solution containing Succ-Ala-Ala-Ala-ρ-nitroaniline (Sigma Co.) was added to a test tube and potassium phosphate buffer and distilled water were added. 0.2 ml of Areca catechu seed extract in ethanol was added to the reactant and then 10 μl of elastase solution were added to react at 37° C. for 10 min. Absorbance was measured at 410 nm for detecting p-nitroaniline released. The control group contains distilled water instead of the extract. Inhibition ratio against elastase activity was calculated by the following equation:
Inhibition ratio to elastase activity (%)=[1−(activity of elastase in the extract-treated group/activity of elastase in the control group)]×100

TABLE IV

| | Final concentration of the | Inhibition ratio to |
|---|---|---|
| Category | extract (μg/ml) | elastase activity (%) |
| Control | 1,000 | 0 |
| | 100 | 0 |
| Areca catechu | 1,000 | 100 |
| seed extract | 100 | 68 |

As demonstrated in Table IV, the Areca catechu seed extract completely inhibited elastase activity at 100% level at a relatively high concentration.

### Experimental Example IV

### Analysis of the Effect on Cell Proliferation

### Experimental Example IV-1

Effect on Keratinocyte Proliferation

1×10⁴ cells of human normal kerationcytes were inoculated into each well of 96-well microplate and cultivated in DMEM for 24 hr. The medium in microplate was replaced by DMEM without serum containing 250 μg/ml of the mixed extract of Glycyrrhiza glabra extract and Areca catechu seed extract in DMSO (dimethyl sulfoxide) and then cultivated for additional 24 hr. Into each well, 10 μl of MTT solution [3-(4,5-dimethyl-thiazole-2-yl) 2,5-diphenyl tetrazolium bromide: 5 mg/ml] were added and allowed to stand for 4 hr, followed by discarding the medium. 100 μl of DMSO were added to each well and agitated for 20 min, after which absorbance at 570 nm was measured by use of microplate Reader. The results are summarized in Table V where the values are average of 3 independent experiments. The effect on cell proliferation was calculated by the following equation:
Cell proliferation effect (%)=[(absorbance of extract-treated group−absorbance of control)/absorbance of control]×100

TABLE V

| Concentration of mixed | Cell proliferation effect |
|---|---|
| extract (μg/ml) | (%) |
| 0 | — |
| 10 | 8.3 |
| 50 | 17.5 |
| 100 | 31.8 |
| 200 | 66.3 |
| 500 | 78.5 |

As demonstrated in Table V, the mixed extract containing Glycyrrhiza glabra extract and Areca catechu seed extract can enhance the proliferation of kerationcyte in dose-dependent manner. Therefore, it could be understood that the mixed extract is very effective in remedying skin wrinkles.

### Experimental Example IV-2

Effect on Fibroblast Proliferation

1×10⁴ cells of human normal fibroblsts were inoculated into each well of 96-well microplate and cultivated in DMEM for 24 hr. The medium in microplate was replaced by DMEM without serum containing 250 μg/ml of the mixed extract of Glycyrrhiza glabra extract and Areca catechu seed extract in DMSO and then cultivated for additional 24 hr. Into each well, 10 μl of MTT solution [3-(4,5-dimethyl-thiazole-2-yl)2,5-diphenyl tetrazolium bromide: 5 mg/ml] were added and allowed to stand for 4 hr, followed by discarding the medium. 100 μl of DMSO were added to each well and agitated for 20 min, after which absorbance at 570 nm was measured by use of microplate Reader. The results are summarized in Table VI where the values are average of 3 independent experiments. The effect on cell proliferation was calculated by the above equation

TABLE VI

| Extract | Cell proliferation effect (%) |
|---|---|
| Areca catechu seed extract | 28.7 |
| Glycyrrhiza glabra extract | 18.2 |
| Glycyrrhiza glabra extract (50%) + Areca | 60.1 |
| catechu seed extract (50%) | |

As indicated in Table VI, each of Glycyrrhiza glabra extract and Areca catechu seed extract can enhance the proliferation of fibroblast that has been known to play an important role in remedy of skin wrinkles and improvement of skin elasticity. In addition, the mixed extract exhibits a synergic effect on the proliferation of fibroblast. The proliferation of fibroblast is closely related to biosynthesis of collagen, elastin, integrin and laminin that are pivotal proteins for remedy of skin wrinkles and improvement of skin elasticity.

### Experimental Example V

### Analysis of the Effect on Integrin Biosynthesis

2×10⁴ cells of human normal fibroblsts were inoculated into each well of 96-well microplate and cultivated in DMEM for 24 hr. The medium in microplate was replaced by DMEM without serum containing 100 μg/ml of Areca catechu seed extract in DMSO and then cultivated for additional 72 hr. The level of integrin was measured by ELISA method. The results are summarized in Table VII.

TABLE VII

| | Effect on increase of |
|---|---|
| Sample | integrin biosynthesis (%) |
| Control | 0 |
| Areca catechu seed extract | 19.47 |
| (100 μg/ml) | |

As shown in Table VII, Areca catechu seed extract can increase the biosynthesis of integrin that has been reported to be a connective protein for promoting a signal transmission between cells and a cell activity by allowing the connection between fibroblasts and other cells.

### Experimental Example VI

### Analysis of Effect of Biosome Containing Mixed Extract on Skin Penetration

The dermal equivalent was placed an inner part of the plate that was sectioned with the membrane of 3 μm porous polycarbonate. The dermal equivalent was prepared as follows: 1×10⁵ cells/ml of human normal fibroblasts were inoculated into the medium containing collagen solution 3 mg/ml: 5×DMEM: 0.05 N sodium hydroxide with 2.2% sodium bicarbonate and 200 mM HEPES buffer, 7:2:1. Then, the cultivation was performed under 5% CO₂ for 7 days at 37° C., thereby obtaining the dermal equivalent.

Onto the surface of the dermal equivalent containing cultivated fibroblasts, 1×10⁵ cells/ml of epidermal kerationcyte were inoculated and then K-SFM medium containing EGF and BPE was added to both the inner and outer parts of the plate, followed by cultivation for 7 days. Thereafter, the medium in the inner part was discarded to contact the cultured cells with air. The medium containing the same volume of K-SFM with 10% FBS and DMEM with no EGF was added into the outer part and the cultivation was performed for 2 weeks so that an artificial skin comprising multi-layered epidermis and dermis was given. Onto the artificial skin, the cosmetic compositions of Example and Comparative Example were applied and then incubated for 4 hr. Then, in the medium containing the same volume of K-SFM with 10% FBS and DMEM with no EGF, the amount of the extracts, that penetrated into the epidermis and dermis of the artificial skin, was analyzed by use of HPLC. The results are summarized in Table VIII.

TABLE VIII

Amount of extract in medium
(μg/ml)

| Type of Extract | Example | Com. Example 1 |
|---|---|---|
| Areca catechu seed extract | 650 | 56 |
| Glycyrrhiza glabra extract | 726 | 69 |

As indicated in Table VIII, the biosome carrying Glycyrrhiza glabra extract and Areca catechu seed extract exhibits better skin penetration, about 10-fold compared to that of Comparative Example 1. This is because an average particle size of the present biosome is about 100 nm, so that its skin penetration occurs feasibly. Such improved penetration ability is responsible for excellent clinical effects of Experimental Examples 7 and 8.

### Experimental Example VII

### Analysis of Effect on Skin Elasticity

The clinical effects of the cosmetic composition containing the biosome carrying Glycyrrhiza glabra extract and Areca catechu seed extract on skin elasticity were analyzed.

30 healthy women (average age of 36.3) were classified into 3 groups and under the conditions of 24-26° C. and 75% humidity, the cosmetic composition of Example was applied to the group A, that of Comparative Example 1 to the group B and that of Comparative Example 2 to the group C. The applications around eyes were continued for 12 weeks, and then the skin elasticity was measured using Cutometer SEM 575 (C+K Electronic Co., Germany). The results were expressed as ΔR8 (R8(12 weeks)-R8(0 week)) of Cutometer SEM 575. R8 values indicate skin viscoelasticity.

TABLE IX

| Sample | Effect on skin elasticity |
|---|---|
| Example | 0.52 |
| Comparative Example 1 | 0.29 |
| Comparative Example 2 | 0.08 |

As demonstrated in Table IX, the effect of Example containing biosome on elasticity is increased 79.3% compared to that of Comparative Example 1. In addition, the composition containing both Glycyrrhiza glabra extract and Areca catechu seed extract, Comparative Example 1, is also effective in skin elasticity.

### Experimental Example VIII

### Analysis of Skin-Whitening Effect

Thirty women aged above 30 were classified to 2 groups. To group A, applied was the formulation comprising the biosome (Example), and to group B the formulation of Comparative Example 1. Applied portion was eye rim and lower part of eye, the time period for application was 12 weeks and applied dose was 0.2 mg per application. After application, anti-shadow effects were measured. The change of skin color (ΔL) was measured by means of chromameter (Minolta CR300). L value indicates the level of lightness, is classified from 0 (black) to 10 (white). In addition to this, the objective evaluation with naked eye was made by a plurality of well-trained testers and the subjective evaluation was made by testee oneself. The evaluation is classified based on the following 7 levels: −3, severe deterioration; −2, deterioration; −1, a little deterioration; 0, no change; +1, a little amelioration; +2, amelioration; and +3, remarkable amelioration.

TABLE X

| | | |
|---|---|---|
| | Objective | |
| | evaluation of | Subjective |
| Change of skin | well−trained | evaluation of |
| lightness (Δ L) | tester | testee |

| Testee | A | B | A | B | A | B |
|---|---|---|---|---|---|---|
| 1 | 5.1 | 1.9 | 1 | 1 | 1 | 0 |
| 2 | 5.6 | 1.0 | 3 | 0 | 3 | 0 |
| 3 | 3.2 | 1.8 | 1 | −1 | 2 | 0 |
| 4 | 7.1 | 0.3 | 2 | 0 | 3 | 0 |
| 5 | 7.4 | 2.1 | 3 | 1 | 2 | 2 |
| 6 | 5.2 | −0.5 | 1 | 0 | 2 | 0 |
| 7 | 5.5 | 1.6 | 3 | 1 | 3 | 1 |
| 8 | 8.8 | 2.2 | 3 | 1 | 3 | 2 |
| 9 | 2.9 | 0.7 | 2 | 1 | 1 | 0 |
| 10 | 5.2 | −1.0 | 2 | 0 | 1 | 1 |
| mean | 5.6 | 1.01 | 2.1 | 0.4 | 2.1 | 0.6 |

As shown in Table X, ΔL value of group A that was subject to the application of the biosome carrying Glycyrrhiza glabra extract and Areca catechu seed extract is 5.6 (p<0.01) and that of group B is 1.01 (p>0.05). Therefore, it could be appreciated that the biosome stabilizing two effective ingredients, Glycyrrhiza glabra extract and Areca catechu seed extract, is very successful in whitening and lighting color tone of skin.

In the objective evaluation, groups A and B show 2.1 (p<0.01) and 0.4 (p>0.05) and in the subjective evaluation, groups A and B show 2.1 (p<0.01) and 0.6 (p>0.05).

Summarizing these results, it will be resulted that the biosome stabilizing Glycyrrhiza glabra extract and Areca catechu seed extract is very reliable and effective in whitening and lighting skin color.

### Experimental Example IX

### Evaluation of Formulation Stability

The formulations of Example and Comparative Example 1 in Table I were stored in an opaque container for 12 weeks in incubator with a constant temperature of 45° C. Independently, the formulations were stored in an opaque container for 12 weeks in a shading refrigerator with a constant temperature of 4° C. Then, the separation and the discoloration levels of the formulations were examined. The separation and the discoloration levels were classified to 6 levels: 0: no change; 1: very slightly discolored (separated); 2: slightly discolored (separated); 3: slightly remarkable discoloration (separated); 4: remarkable discoloration (separated); and 5: very remarkable discoloration (separated).

TABLE XI

Discoloration (separation) level

| Temp. | Example | Com. Example 1 |
|---|---|---|
| 45° C. | 0 | 2.0 |
| 4° C. | 0 | 0 |

As indicated in Table XI, it will be appreciated that the cream formulation containing the biosome (Example) is stable one without showing discoloration and separation. The formulation of Comparative Example 1, showed a little discoloration and separation at 45° C.

### Experimental Example X

### Evaluation of Safety on Skin

30 testees (mean age, 27.5, age range 19-35) were classified to 2 groups and skin patch test was carried out using Haye's Test Chamber. Persons, who showed symptoms such as psoriasis, eczema and other skin lesions, pregnant women, breast-feeding women and persons taking contraceptive or antihistamine drug were excluded from this test.

15 g of the formulations in Table I were dropped into the Chamber and the Chamber was fixed on the tested portion, upper arm that had been washed with 70% ethanol and dried. To group A, applied was the formulation comprising the biosome (Example), and to group B the formulation of Comparative Example 1. After 24 hr application, the patch was detached and the tested portion was marked with a marking pen. After 24 hr or 48 hr, the tested portion was observed. The skin response was determined according to the criteria in Table XI provided by International Contact Dermatitis Research Group (ICDRG). The results are summarized in Table XII.

TABLE XI

| [symbol] | Criteria | Evaluation | Mean Score |
|---|---|---|---|
| ± | Doubtful or slight reaction and | Very Slight | 0-0.9 |
| | erythema | irritation | |
| + | Erythema + Induration | Slight | 1.0-2.9 |
| | | irritation | |
| ++ | Erythema + Induration + Vesicle | Moderate | 3.0-4.9 |
| | | irritation | |
| +++ | Erythema + Induration + Bullae | Strong | above 5.0 |
| | | irritation | |
| − | Negative | no | 0 |
| | | irritaion | |

TABLE XII

| Time after detach of | | |
|---|---|---|
| patch | Example | Comparative Example 1 |
| 24 hr | 0 | 0.2 |
| 48 hr | 0 | 0 |

As shown in Table XII, it could be appreciated that the Example formulation comprising biosome carrying Glycyrrhiza glabra extract and Areca catechu seed extract exhibits excellent safety one to skin since it shows little or no skin irritation. The formulation of Comparative Example 1 also shows safety to skin since its effective substances are extracts from plant.

### FORMULATION EXAMPLES

Based on the results from the Experimental Examples, the cosmetic compositions comprising biosome stabilizing Glycyrrhiza glabra extract and Areca catechu seed extract were prepared. It is understood that the present formulation is not limited to the following specific examples and that variants and modifications may become apparent to those skilled in the art.

### Formulation Example I

### Skin Softener (Skin Lotion)

| Ingredients | Amount (wt %) |
|---|---|
| Biosome | 1.0 |
| 1,3-butylene glycol | 6.0 |
| Glycerine | 4.0 |
| Oleyl alcohol | 0.1 |
| Polysorbate 20 | 0.5 |
| Ethanol | 15.0 |
| benzophenone-9 | 0.05 |
| Perfume, preservative | 0.3 |
| Distilled water | To 100 |

### Formulation Example II

### Nutrient Liquid (Milk Lotion)

| Ingredients | Amount (wt %) |
|---|---|
| Bisome | 3.0 |
| Propylene glycol | 6.0 |
| Glycerine | 4.0 |
| Triethanol amine | 1.2 |
| Tocopheryl acetate | 3.0 |
| Liquid paraffin | 5.0 |
| Squalane | 3.0 |
| Makadamianut oil | 2.0 |
| Polysorbate 60 | 1.5 |
| Sorbitan sesquinolate | 1.0 |
| Carboxyvinyl polymer | 1.0 |
| BHT | 0.01 |
| EDTA-2Na | 0.01 |
| Perfume, preservative | 0.3 |
| Distilled water | to 100 |

### Formulation Example III

### Nutrient Cream

| Ingredients | Amount (wt %) |
|---|---|
| Biosome | 10.0 |
| Cetosteary alcohol | 2.0 |
| Glycerol stearate | 1.5 |
| Trioctanoine | 5.0 |
| Polysorbate 60 | 1.2 |
| Sorbitan stearate | 0.5 |
| Squalane | 5.0 |
| Liquid paraffin | 3.0 |
| Cyclomethicon | 3.0 |
| BHT | 0.05 |
| Delta-tocopherol | 0.2 |
| Conc. glycerine | 4.0 |
| 1,3-butylene glycol | 2.0 |
| Santan gum | 0.1 |
| EDTA-2Na | 0.05 |
| Perfume, preservative | 0.3 |
| Distilled water | to 100 |

### Formulation Example IV

### Massage Cream

| Ingredients | Amount (wt %) |
|---|---|
| Biosome | 1.0 |
| Propylene glycol | 6.0 |
| Glycerine | 4.0 |
| Triethanol amine | 0.5 |
| Wax | 2.0 |
| Tocopheryl acetate | 0.1 |
| Polysorbate 60 | 3.0 |
| Sorbitan sesquinoleate | 2.5 |
| Cetearyl alcohol | 2.0 |
| Liquid paraffin | 30.0 |
| Carboxyvinyl polymer | 0.5 |
| Perfume, preservative | 0.4 |
| Distilled water | to 100 |

### Formulation Example V

### Pack

| Ingredients | Amount (wt %) |
|---|---|
| Biosome | 2.0 |
| Propylene glycol | 2.0 |
| Glycerine | 4.0 |
| Carboxyvinyl polymer | 0.3 |
| Ethanol | 7.0 |
| PEG-40 hydrogenated | 0.8 |
| castor oil | |
| Triethanol amine | 0.3 |
| BHT | 0.01 |
| EDTA-2Na | 0.01 |
| Perfume, preservative | 0.4 |
| Distilled water | to 100 |

Having described a preferred embodiment of the present invention, it is to be understood that variants and modifications thereof falling within the spirit of the invention may become apparent to those skilled in this art, and the scope of this invention is to be determined by appended claims and their equivalents.

What is claimed is:

1. A composition for promoting fibroblasts and karatinocytes proliferation, which comprises an extract from a seed of Areca catechu and an extract from Glycyrrhiza glabra.

2. A cosmetic composition with dual function for skin whitening and remedy of skin wrinkles comprising: (a) an extract from a seed of Areca catechu and an extract from Glycyrrhiza glabra as an active ingredient; and (b) a cosmetically acceptable carrier.

3. The cosmetic composition according to claim 2, wherein each of said extract from the seed of Areca catechu and said extract from Glycyrrhiza glabra is present in an amount of 0.001-1.0 wt % based on the total weight of said composition.

4. The cosmetic composition according to claim 2, wherein said extract from the seed of Areca catechu and said extract from Glycyrrhiza glabra is entrapped into a biosome.

5. The cosmetic composition according to claim 4, wherein said biosome is present in an amount of 0.05-20 wt % based on the total weight of said composition.

6. The cosmetic composition according to claim 4, wherein said extract from the seed of Areca catechu and said extract from Glycyrrhiza glabra is entrapped into said biosome with an amount of 0.01-5.0 wt % based on the total weight of said biosome.

7. The cosmetic composition according to claim 4, wherein said biosome is prepared by use of a non-ionic surfactant and a ceramide bound to glycerin.

8. The cosmetic composition according to claim 7, wherein said ceramide is a pseudoceramide represented by the following formula (I): 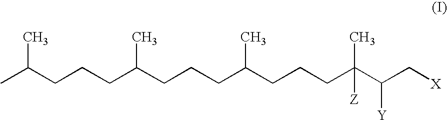
wherein Z represents —OH and Y represents —OH, 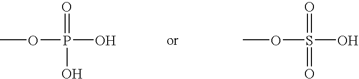
with the proviso that X is 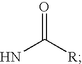
Z represents —OH and X represents —OH, 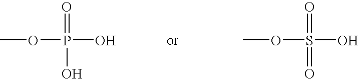
with the proviso that Y is 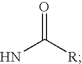
Y represents —OH and X represents —OH, 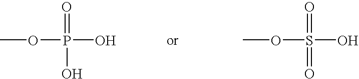
with the proviso that Z is 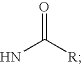
R represents a linear or branched, saturated or unsaturated aliphatic hydrocarbon group; and when substituted, R has one or more —OH groups.

9. The cosmetic composition according to claim 8, wherein said pseudoceramide represented by the following formula (II), (III) or (IV): 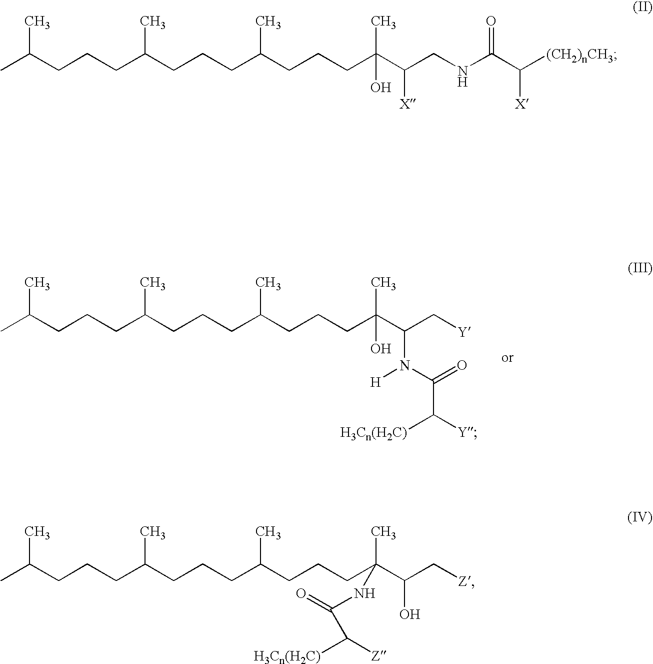
wherein X′ represents H or —OH; X″ represents —OH, 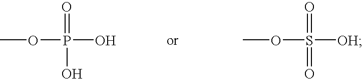
Y′ represents —OH, 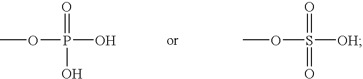
Y″ represents H or —OH; Z′ represents —OH, 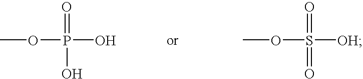
Z″ represents H or —OH; and n is 0 or an integer of from 1 to 47.

10. The composition according to claim 2, wherein said cosmetic composition is in the form of one selected from the group consisting of a solution, a suspension, an emulsion, a paste, an ointment, a gel, a cream, a lotion, a powder, a soap, a surfactant-containing cleanser, an oil, a powder foundation, an emulsion foundation, a wax foundation and a spray.
